(19) United States
(12) Patent Application Publication
(10) Pub. No.: US 2016/0257930 A1
(43) Pub. Date: Sep. 8, 2016

(54) NEURONAL STEM CELL DIFFERENTIATION

(71) Applicants: Paul KEMP, Cardiff, South Glamorgan (GB); Nicholas ALLEN, Cardiff, South Glamorgan (GB)

(72) Inventors: Paul Kemp, Cardiff (GB); Nicholas Allen, Cardiff (GB)

(21) Appl. No.: 15/029,164

(22) PCT Filed: Oct. 13, 2014
(86) PCT No.: PCT/GB2014/053064

§ 371 Date: Apr. 13, 2016

(30) Foreign Application Priority Data
Oct. 14, 2013 (GB) 1318126.8

(51) Int. Cl. C12N 5/0793

(52) U.S. Cl. CPC C12N 5/0619; C12N 2501/40; C12N 2506/08

(57) ABSTRACT

The invention relates to at least one novel neural cell differentiation medium for the production of neural cells from at least one precursor cell capable of lineage specific differentiation; a method for neural cell differentiation using said medium; the use of said medium and said method for lineage specific differentiation of at least one precursor cell towards a neural cell fate; and a kit of parts comprising said at least one said neural cell differentiation medium.

### FIELD OF THE INVENTION

The invention relates to at least one novel neural cell differentiation medium for the production of neural cells from at least one precursor cell capable of lineage specific differentiation; a method for neural cell differentiation using said medium; the use of said medium and said method for lineage specific differentiation of at least one precursor cell towards a neural cell fate; and a kit of parts comprising said at least one said neural cell differentiation medium.

### BACKGROUND OF THE INVENTION

Stem cells are biological cells found in all multicellular organisms, which can divide and differentiate into diverse specialized cell types and can self-renew to produce more stem cells. In mammals, there are two naturally occurring broad types of stem cells: embryonic stem cells, which are isolated from the inner cell mass of blastocysts, and adult stem cells, which are found in various tissues. There is also a third class of stem cells which are man-made and known as induced pluripotent stem cells (iPSCs). iPSCs are made by de-differentiating adult cells to a stem cell like phenotype. In adult organisms, stem cells and progenitor cells act as a repair system for the body, replenishing adult tissues. In a developing embryo, stem cells can differentiate into all the specialized cells (they are pluripotent cells), but they also maintain the normal turnover of regenerative organs, such as blood, skin, or intestinal tissues.

Embryonic and somatic stem cells have the ability to differentiate into any cell type; they are therefore uniquely suited for cell replacement therapies for diseases which destroy, or damage/injure, a defined cell population. The unique pluripotency of stem cells provides them with the potential to regenerate themselves and also to develop into the various cell types of all three embryonic germ layers, i.e. ectoderm, mesoderm and endoderm, under appropriate developmental (or culture) conditions. The potential of ESCs, induced pluripotent stem cells (iPSCs), adult or tissue specific stem cells and the like to grow into specialized cells has attracted interest from researchers wishing to develop new treatments using these cells.

Regenerative medicine is focussed on regenerating damaged tissues and organs in the body by replacing damaged tissue and/or by stimulating the body's own repair mechanisms to heal previously irreparable tissues or organs. This often involves the use of cells or tissue in cell or tissue therapy. There are many potential forms of cell therapy, including: the transplantation of stem cells or progenitor cells that are autologous (from the patient) or allogeneic (from another donor); the transplantation of mature, functional cells (Cell Replacement Therapy); and the application of modified human cells that are used to produce a needed substance (cell-based gene therapy). Commonly, cells or tissues utilised in cell therapy are either those obtained from the same individual, another donor individual, or alliteratively, grown and developed in vitro in laboratories. The generation of iPSCs from differentiated somatic cells offers great promise as a possible means for treating diseases through cell transplantation. The possibility of generating iPSCs cells from somatic cells derived from individual patients also may enable the development of patient-specific therapies with less risk of immune rejection.

Another application of these cells involves differentiating large quantities of stem cells into particular human cell types and using these cells for pharmaceutical drug testing or toxicity testing. Lineage-specific differentiated stem cells are therefore also valuable research tools for a variety of purposes including in vitro screening assays, elucidation of the complex mechanisms of cell lineage specification and differentiation, and identifying critical biochemical differences between normal and diseased or damaged states, differences which can be exploited for use as diagnostic or prognostic markers.

The power of embryonic and somatic stem cells as therapeutics and model systems for neurodegenerative diseases has been well explored. Reconstruction of neural function in advanced neurodegenerative conditions such as Alzheimer's disease, Parkinson's disease, Huntington's disease, Amyotrophic lateral sclerosis and the like requires replacement of the neural cells lost by cell death. iPSCs from human patients with well-defined, and often genetically determined neuronal pathology, have huge potential both for disease modelling and for reliable high-throughput drug screening.

Various factors and molecules regulate the differentiation of stem cells into neurons, for example, epidermal growth factor (EGF) and fibroblast growth factor-2 (FGF-2) promote proliferation while retaining the cells in an undifferentiated state. In addition, insulin and insulin like growth factor 1 (IGF1) have also been shown to be essential for neural stem cell growth and proliferation. Retinoic acid, insulin, Ifg1, Nogo-66, LPA (lysophosphatidic acid), PTEN (phosphatase and tensin homolog) and Bone morphogenetic protein (BMP)-4 are known to control the differentiation of stem cells into neurons or astrocytes. Pluripotent stem cells (PSCs) differentiate into neurons through a program of neuralisation, neuronal-subtype fate specification, cell cycle exit, post-mitotic neuronal differentiation and functional maturation of regulated excitability.

Due to this complex development, the consistent and reproducible generation of functional neurons from stem cells is crucial to the development of cellular models of neurological disease. A number of protocols for neuronal differentiation have been proposed. For example, WO2004/046348 teaches differentiation protocols for the generation of neural-like cells from bone marrow-derived stem cells. WO2006/134602 teaches differentiation protocols for the generation of neurotrophic factor secreting cells. WO2007/066338 teaches differentiation protocols for the generation of oligodendrocytic-like cells. WO2010/090843 discloses gingiva-derived mesenchymal stem cells and their use in immunomodulation and reconstruction.

However, whilst numerous protocols for neuronal differentiation are known to produce neuronal models, only a few provide robust or reliable measures of functional neuronal maturation and synaptogenesis. This therefore limits the utility of the differentiated cells for both disease modelling and the development of novel therapies in vitro, because despite sharing similarity with neuronal cells, many of the existing protocols do not generate neuronal cells that are fully functional and faithfully replicate those cells obtained in vivo. Many existing protocols rely on molecular and cellular markers as the primary measure of effectiveness; for example, β-III-Tubulin and microtubule-associated protein (MAP) 2 together with markers of neuronal subtype-specific fate determination. Such markers are useful in determining how neurogenic each protocol might be, but they give very little indication as to whether the differentiated cells express the critical characteristics of functional neurons. Optimal use of patient-derived, stem cells for modelling neuronal diseases is crucially dependent upon the proper physiological maturation of derived neurons.

Further, despite significant advances in the development of protocols for stem cell neuralisation and neural progenitor fate-specification, the latter stages of neural differentiation are still difficult to control. Notably, developmental studies are confounded by on-going progenitor proliferation and neurogenesis in long-term cultures. Moreover, the protracted time in culture required for differentiated neurons to become functionally mature often exceeds 100 days.

The present invention relates to the field of stem cell biology, in particular the neuronal differentiation of neural progenitors derived from pluripotent or multipotent stem cells, which can include, but is not limited to, human embryonic stem cells (hESC), human induced pluripotent stem cells (hiPSC), adult stem cells, somatic stem cells, cancer stem cells, or any other cell capable of lineage specific differentiation. It is herein disclosed a novel method and cell culture medium to control stem cell differentiation, and in particular neural stem cell differentiation. In contrast to existing techniques, which can often take several months, advantageously, it has been found that the disclosed method and media results in maturation of stem cell-derived neural progenitors to produce a high yield of functional, spontaneously electrically excitable neurons within 2-3 weeks. Moreover, the method and media has excellent fidelity resulting in almost 100% efficiency in neuron differentiation by 4 weeks.

The invention therefore provides a simple and convenient way to promote and accelerate the functional maturation of stem cell derived neurons in vitro. Efficacious and accelerated controlled differentiation of human or animal stem cells into functional neuronal cells will reduce culture times and thereby costs, whilst also generating cells that more accurately reflect those existing in vivo. This will allow experimental dissection of the events during early development of the nervous system, and the identification of new therapeutic targets. Additional pharmaceutical applications may include the creation of new assays for toxicology and drug discovery, such as high-throughput screens for neuro-protective compounds. The generation of neural cells from stem cells in vitro may serve as an unlimited source of neural cells for tissue reconstruction and for the delivery and expression of genes in the nervous system.

### STATEMENTS OF INVENTION

According to a first aspect of the invention there is provided a neural cell differentiation medium for the production of neural cells from at least one precursor cell capable of lineage specific differentiation comprising, or consisting of,
i) at least one cell culture base medium;
ii) at least one serum-free supplement for neural cell culture;
iii) a cell-cycle inhibitor;
iv) at least one Tropomyosin receptor kinase B [TrkB] receptor agonist;
v) a canonical Wnt pathway agonist;
vi) ascorbic Acid or vitamin C or a salt or derivative thereof; and
vii) a solution of calcium ions.

Reference herein to a neural cell differentiation medium refers to a cell culture medium for use in the culture and growth of cells and to control cell differentiation, and in particular lineage specific differentiation of a neural precursor cell to a neural cell wherein said precursor is generated from pluripotent or multipotent stem cells such as but not limited to, embryonic stem cells (ESC), induced pluripotent stem cells (iPSC), adult stem cells, somatic stem cells, cancer stem cells, progenitor cells or any other cell capable of lineage specific differentiation into a neural cell. A neural cell is to be construed as any cell type classified as part of the nervous system, such as but not limited to, neurones, astrocytes or glial cells.

Reference herein to a cell culture base medium includes reference to a liquid or gel designed to support the growth of precursor cells, in particular stem cells or progenitor cells. As will be appreciated by those skilled in the art, many different types of chemical medium can be used to support the growth of stem or progenitor cells in culture, such as but not limited to, Dulbecco's Modified Eagle Medium (DMEM), DMEM: Nutrient Mixture F-12 (DMEM/F-12), Stemline (SL) and Neurobasal (NB).

In a preferred embodiment of the invention, said cell culture base medium is DMEM or DMEM:F12 base medium, or Advanced DMEM:F12 a formulation with added serum-free supplement. Ideally, said cell culture base medium is Advanced DMEM: F12.

Reference herein to a serum free supplement is to a supplement that supports, encourages, drives, promotes or sustains neural cells in culture but does not contain any detectable serum or components thereof. Examples include, but not limited to, N2 supplement, B27 supplement and NeuroBrew21™, or the like.

Reference herein to a cell-cycle inhibitor is to any compound that slows, arrests or stops cell cycle progression. As is known by those skilled in the art, cell cycle arrest can be induced at different stages of the cell cycle, for example at the G1-S, G2-M or M cellular checkpoints, decreasing the rate of cell division and the number of actively cycling cells. Small molecule examples of cell cycle inhibitors are well known to those skilled in the art, such as but not limited to, ABT751, AZD5438, Baicalein, 8-chloroadenosine, CI 898 trihydrochloride, Daidzein, DIM, Epothilone B, Indirubin-3′-oxime, Methotrexate, Narciclasine, Plumbagin, SKPin C1, WYE 687 dihydrochloride, YC 1, flavopiridol, olomoucine, indisulam, SNS-032, bryostatin-1, selicidib, PD 0332991, mimosine, deferoxamine, purvalanol, SCH 727965, or the like.

In a preferred embodiment of the invention, said cell-cycle inhibitor is an inhibitor of cell cycle progression targeting the G1-S phase checkpoint, such as but not limited to, PD0332991, AZD5438, Baicalein, CI 898 trihydrochloride, Daidzein, WYE 687 dihydrochloride, YC 1, flavopiridol, indisulam, purvalanol, seliciclib, mimosine, purvalanol.

Most preferably, said cell-cycle inhibitor is PD0332991.

However, the invention also encompasses cell-cycle inhibitors that are biomolecules such as isolated or recombinant peptides, proteins or antibodies, or the like such as, although not limited to, DQ 65-79, TGFbeta, activin, and the anti-HER2 antibody trastuzumab.

Reference herein to a Tropomyosin receptor kinase B [TrkB] receptor agonist is any compound that promotes or increases TrkB receptor activity, the high affinity catalytic receptor for several “neurotrophins”, which are small protein growth factors that induce the survival and differentiation of distinct cell populations. Such compounds include recombinant proteins of the neurotrophins e.g. Brain Derived Neurotrophic Factor (BDNF), neurotrophin-4 (NT-4), Glial cell-derived neurotrophic factor (GDNF) and neurotrophin-3 (NT-3), and analogues thereof, and small molecule TrkB agonists, such as but not limited to, 7,8-dihydroxyflavone, N,N′,N″Tris(2-hydroxyethyl)-1,3,5-ben-zenetricarboxamide (LM22A4), 4′-dimethylamino-7,8-dihydroxyflavone, or the like.

In a preferred embodiment of the invention, said TrkB receptor agonists is recombinant BDNF or LM22A4.

However, the invention also encompasses Tropomyosin receptor kinase B [TrkB] receptor agonists that are biomolecules such as isolated or recombinant peptides, proteins or antibodies which agonise TrkB receptors, such as, but not limited to, TAM-163, or the like.

Reference herein to a canonical Wnt pathway agonist is any compound that promotes or activates the canonical Wnt signaling pathway. As is known by those skilled in the art, the canonical Wnt pathway (or Wnt/β-catenin pathway) is the Wnt pathway that causes an accumulation of β-catenin in the cytoplasm and its eventual translocation into the nucleus to act as a transcriptional co-activator of transcription factors that belong to the TCF/LEF family.

In a preferred embodiment of the invention, said canonical Wnt pathway agonist is a Glycogen synthase kinase 3 beta (GSK3β) inhibitor. As is known by those skilled in the art, GSK-3 has been shown to phosphorylate Beta-catenin, thus targeting it for degradation and inhibitors of this protein are known to specifically promote non-canonical Wnt signaling. Such compounds include, but are not limited to, CHIR99021, (2′Z,3′E)-6-Bromoindirubin-3′-oxime (BIO), (2′Z,3′E)-6-Bromoindirubin-3′-acetoxime (BIO-acetoxime), 5-Ethyl-7,8-dimethoxy-1H-pyrrolo[3,-4-c]isoquinoline-1,3(2H)-dione (3F8), 1-(7-Methoxyquinolin-4-yl)-3-[6-(tr-ifluoromethyl)pyridin-2-yl]urea (A1070722), N-[(4-Methoxyphenyl)methyl]-N′-(5-ni-tro-2-thiazolyl)urea (AR-A 014418), 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrole-2,5-dione (SB415286), N-(3-Chloro-4-methylphenyl)-5-(4-ni-trophenyl)-1,3,4-oxadiazol-2-amine (TC-G24), 2-Methyl-5-[3-[4-(methylsulfinyl)ph-enyl]-5-benzofuranyl]-1,3,4-oxadiazole (TCS2002), 3-[[6-(3-Aminophenyl)-7H-pyrrolo[2,-3-d]pyrimidin-4-yl]oxyphenol ditrifluoroacetate (TWS 119), or the like.

Most ideally, said canonical Wnt pathway agonist is CHIR99021.

Reference herein to ascorbic acid refers to an organic compound constituting one form of Vitamin C, an important cofactor in many enzymatic reactions. However, as will be appreciated by those skilled in the art, alternative forms of Vitamin C and derivatives thereof could be utilized in accordance with the invention.

Reference herein to a solution of calcium ions refers to any chemical solution comprising free Ca2⁺ ions, most preferably Calcium Chloride.

In yet a further preferred embodiment of the invention, said neural cell differentiation medium additionally, or alternatively, further comprises, or consists of, at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

Reference herein to a notch signaling inhibitor is any compound that antagonizes or inhibits the notch signaling cell pathway, which is known to promote cell cycle exit and regulate proneurogenic gene expression.

In a preferred embodiment of the invention, said notch signaling inhibitor is a γ-secretase inhibitor, such as but not limited to N-[N-(3,5-Difluorophenacetyl)-L-alanyl]-S-phenylglycine t-butyl ester (DAPT), Semagacestat, BMS-299897, BMS-708163, dibenzazepine, MK-0752, RO4929097, LY900009, LY3039478, LY411575, MRK-003, MRK-006, BMS-906024, or the like.

Most preferably said notch signaling inhibitor is DAPT, Semagacestat, BMS-299897, or BMS-708163.

Reference herein to CREB activator is any compound that promotes phosphorylation of CREB, a cellular transcription factor which binds to cAMP response elements (CRE), thereby increasing or decreasing the transcription of the downstream genes.

As is known to those skilled in the art, CREB phosphorylation can be achieved through numerous means, such as but not limited to, activation of adenylyl cyclase, the use of cAMP or analogues thereof, or inhibitors of phosphodiesterase. However, any suitable compound that up- or down-regulates a signaling pathway that increases phosphorylation of CREB can be used to the same effect.

In a preferred embodiment of the invention, said CREB activator is an adenylyl cyclase activator such as, but not limited to forskolin, N,N-Dimethyl-(3R,4aR,5S,6aS,10S,10a-R,10bS)-5-(acetyloxy)-3-ethenyldodecahydro-10,10b-dihydroxy-3,4a,7,7,10a-pentamethyl-1-oxo-1H-naphth-o[2,1-b]pyran-6-yl ester β-alanine hydrochloride, Pituitary Adenylate Cyclase-Activating Polypeptide 1-27, 1,9-Dideoxyforskolin, 5-[[2-(6-Amino-9H-purin-9-yl)ethyl]amino]-1-pentanol, Colforsin dapropate hydrochloride, or the like.

Most ideally, said CREB activator is forskolin.

Alternatively, said CREB activator is cAMP or an analogues thereof, or an inhibitor of phosphodiesterase which cleaves the phosphodiester bond in cAMP regulating the localization, duration, and amplitude of cyclic nucleotide signaling within subcellular domains.

Examples of cAMP analogues include Dibutyryl-cAMP, 8-(4-Chlorophenylthio)-2′-O-methylad-enosine-3′,5′-cyclic monophosphate acetoxymethyl ester, 8-Bromo-cAMP, or (S)-Adenosine, cyclic 3′,5′-(hydrogenphosphorothioate) triethylammonium, or the like.

Examples of phosphodiesterase inhibitors include CGH 2466 dihydrochloride, Cilostamide, Cilostazol, 5-[3-[(1S,2S,4R)-Bicyclo[2.2.1]hept-2-yloxy]-4-methoxyphenyl]tetrahydro-2(1H)-pyrimid-inone, Dipyridamole, EHNA hydrochloride, Enoximone, Etazolate hydrochloride, Gisadenafil besylate, MDL 12330A hydrochloride, Mesopram, Rolipram, Pentoxifylline, Anagrelide hydrochloride, N,N,2-Trimethyl-5-nitro-benzenesulf-onamide, 4-[(2R)-2-[3-(Cyclopentyloxy)-4-met-hoxyphenyl]-2-phenylethyl]-pyridine hydrochloride, Vinpocetine, Zaprinast, Zardaverine, or the like.

Reference herein to a γ-aminobutyric acid (GABA) receptor agonist is any compound that binds and activates the GABA receptor, an ionotropic receptor and ligand gated ion channel which upon activation results in hyperpolarization of the neuron.

In a preferred embodiment of the invention, said γ-aminobutyric acid (GABA) receptor agonist includes, but is not limited to, recombinant γ-aminobutyric acid (GABA), Calcium acetylhomotaurinate, 1-(4-Aminobutanoyl)-4-[1,3-bis(dihy-droxyphosphoryloxy)propan-2-yloxy]-7-nitroindoline-, Isoguvacine hydrochloride, 3-(2,5-Difluorophenyl)-7-(1,1-dimet-hylethyl)-6-[(1-methyl-1H-1,2,4-triazol-5-yl)metho-xy]-1,2,4-triazolo[4,3-b]pyridazine, MK0343, MRK 016, Muscimol, trans-4-Aminocrotonic acid, 5-Nitro-α-oxo-N-(1R)-phenylethyl]-1H-indole-3-acetamide, 2′,4-Difluoro-5′-[8-fluoro-7-(1-hydro-xy-1-methylethyl)imidazo[1,2-a]-pyridin-3-yl]-[1,1-′-biphenyl]-2-carbonitrile, (Z)-3-[(Aminoiminomethyl)thio]prop-2-enoic acid sulfate, or the like.

Most ideally, said GABA receptor agonist is GABA.

Reference herein to a non-canonical Wnt pathway agonist is any compound that promotes or activates the non-canonical Wnt/Calcium signaling pathway. As is known by those skilled in the art, non-canonical Wnt/Calcium pathways are those Wnt signaling pathways that do not involve β-catenin and are involved in regulation of intracellular calcium levels.

In a preferred embodiment of the invention, said non-canonical Wnt pathway agonist is a Protein Kinase C (PKC) activator including, but are not limited to, Phorbol 12-myristate 13-acetate (PMA), Phorbol 12,13-dibutyrate, cholesterol sulphate, Bryostatin 1,2-[(2-Pentylcyclopropyl)methyl]cycl-opropaneoctanoic acid (FR 236924), (2S,5S)-1,2,4,5,6,8-Hexahydro-5-(hy-droxymethyl)-1-methyl-2-(1-methylethyl)-3H-pyrrolo-[4,3,2-gh]-1,4-benzodiazonin-3-one, Ingenol 3-angelate, 5-Chloro-N-heptylnaphthalene-1-sulf-onamide, 5-Chloro-N-(6-phenylhexyl)-1-naphth-alenesulfonamide, or the like.

Most ideally, said non-canonical Wnt pathway agonist is PMA.

In a second aspect of the invention, there is provided an ex vivo culture medium comprising, or consisting of,
i) at least one cell culture base medium;
ii) at least one serum-free supplement for neural cell culture;
iii) a cell-cycle inhibitor;
iv) at least one Tropomyosin receptor kinase B [TrkB] receptor agonist;
v) a canonical Wnt pathway agonist;
vi) ascorbic Acid or vitamin C or a salt or derivative thereof; and
vii) a solution of calcium ions.

In yet a further preferred embodiment of the second aspect of the invention, said ex vivo culture medium additionally, or alternatively, further comprises, or consists of at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

This further aspect of the invention may, in preferred embodiments, include or be characterised by any of the afore mentioned features.

In a third aspect of the invention, there is provided a method for neural cell differentiation comprising: culturing at least one precursor cell capable of lineage specific differentiation in a neural cell differentiation media comprising, or consisting of,
i) at least one cell culture base medium;
ii) at least one serum-free supplement for neural cell culture;
iii) a cell-cycle inhibitor;
iv) at least one Tropomyosin receptor kinase B [TrkB] receptor agonist;
v) a canonical Wnt pathway agonist;
vi) ascorbic Acid or vitamin C or a salt or derivative thereof; and
vii) a solution of calcium ions.

More preferably, said precursor cell is selected from the group comprising: immature neural cells or neural progenitor cells, human embryonic stem cells (hESC), human induced pluripotent stem cells (hiPSC), adult stem cells, somatic stem cells, or cancer stem cells.

In a further a preferred embodiment of the third aspect of the invention, said method includes culturing said cell(s) in a neural cell differentiation medium additionally further comprising, or consisting of at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

In yet a further preferred embodiment of the third aspect of the invention, said method comprises:
culturing said neural progenitor cell(s) in a first neural cell differentiation medium comprising i-x;
followed by culturing said cell(s) in a second neural cell differentiation medium comprising i-vii plus xi.

In preferred embodiment of the third aspect of the invention, said method comprises culturing said cell(s) in the first neural cell differentiation medium for at least 1-20 days, or more ideally 3-14 days, or more ideally still a number of days selected from the group comprising or consisting of the following days 3, 4, 5, 6, 7, 8, 9, 10, 11, 12, 13 and 14 days.

Additionally, in yet a further preferred embodiment of the third aspect of the invention, said method comprises culturing said cells in the second neural cell differentiation medium for at least 1-30 days, although those skilled in the art will appreciate the cells can be held in culture for more than 30 days. Ideally, said cells are cultured in the second neural cell differentiation medium for 3-30 days, although those skilled in the art will appreciate the cells can be held in culture for more than 30 days. More ideally still, said cells are cultured in the second neural cell differentiation medium for a number of days selected from the group comprising or consisting of the following days 3, 4, 5, 6, 7, 8, 9, 10, 11, 12, 13, 14, 15, 16, 17, 18, 19, 20, 21, 22, 23, 24, 25, 26, 27, 28, 29 and 30 days, or more.

This further aspect of the invention may, in preferred embodiments, include or be characterised by any of the afore mentioned features.

According to a yet a further aspect of the invention, there is provided the use of a neural cell differentiation medium comprising, or consisting of,
i) at least one cell culture base medium;
ii) at least one serum-free supplement for neural cell culture;
iii) a cell-cycle inhibitor;
iv) at least one Tropomyosin receptor kinase B [TrkB] receptor agonist;
v) a canonical Wnt pathway agonist;
vi) ascorbic Acid or vitamin C or a salt or derivative thereof; and
vii) a solution of calcium ions.
   for lineage specific differentiation of at least one precursor cell into a neural cell.

Most preferably said precursor cell is selected from the group comprising: pluripotent or multipotent stem cells or progenitor cells, such as but not limited to, embryonic stem cells (ESC), induced pluripotent stem cells (hiPSC), adult stem cells, somatic stem cells, cancer stem cells, or any other cell capable of lineage specific differentiation into a neural cell. A neural cell is to be construed as any cell type classified as part of the nervous system, such as but not limited to, neurones, astrocytes or glial cells.

In yet a further preferred embodiment of this further aspect of the invention, said neural cell differentiation medium additionally, or alternatively, further comprises, or consists of at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

In a preferred embodiment of this further aspect of the invention, said neural cell differentiation medium is used to make, propagate or grow neurons.

According to yet a further aspect of the invention there is provided a kit for the culture and/or differentiation of at least one precursor cell capable of lineage specific differentiation into a neural cell comprising:
a. a neural cell differentiation medium comprising, or consisting of,
   i) at least one cell culture base medium;
   ii) at least one serum-free supplement for neural cell culture;
   iii) a cell-cycle inhibitor;
   iv) at least one Tropomyosin receptor kinase B [TrkB] receptor agonist;
   v) a canonical Wnt pathway agonist;
   vi) ascorbic Acid or vitamin C or a salt or derivative thereof; and
   vii) a solution of calcium ions.
b. optionally, reagents and instructions pertaining to the use of said neural differentiation medium.

In a further preferred embodiment of this further aspect of the invention, said neural cell differentiation medium additionally, or alternatively, further comprises, or consists of at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

In yet a further preferred embodiment of the invention, said kit comprises:
a. a first neural cell differentiation medium comprising i-x;
b. a second neural differentiation medium comprising i-vii plus xi; and
c. optionally, reagents and instructions pertaining to the use of said first and second neural differentiation media.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The present invention will now be described by way of example only with particular reference to the following figures wherein:

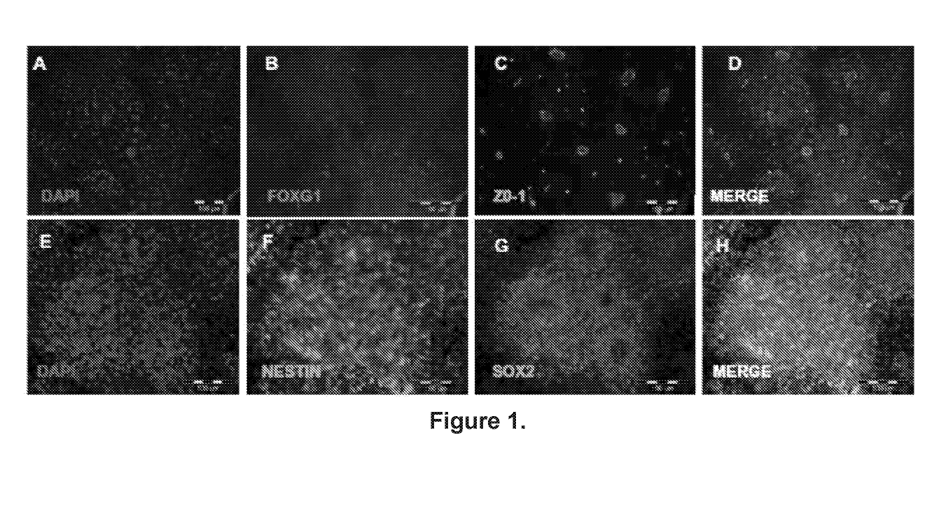

FIG. 1. Efficient neuralisation of human iPS cells. Human iPS cells were neuralised to rosettes using SB431542 and LDN193189 treatment, to inhibit TGFbeta signalling via a dual SMAD inhibition protocol. Specifically, human iPS cells were grown on Matrigel coated plates in mTesR culture medium (Stem Cell Technologies). When 60-70% confluent, cells were washed with PBS and cultured in SLI differentiation medium (Advanced DMEM:F12 supplemented with 2% B27 without retinoic acid and 10 microM SB431542, 1 microM LDN193189 and 1.5 microM IWR1 (all from Tocris) for 8 days. Thereafter, cells were cultured in the same medium, but without SB431542 (SL medium). At 16 days, neural rosettes were fixed and processed for immumocytochemistry.

Left panels, upper (A) and lower (E) show nuclear staining with DAPI in blue. Upper panels show characteristic neural rosette structures, as identified by ZO-1 localisation (C, green) to the centres of each FOXG1-positive rosette (B, red). D, merge of B and C to illustrate rosette structure.

Lower panels show co-expression of neural progenitor markers Nestin (F, green) and Sox2 (G, red). H is a merge of F and G.

Scale bars represent 100 micrometres, as indicated.

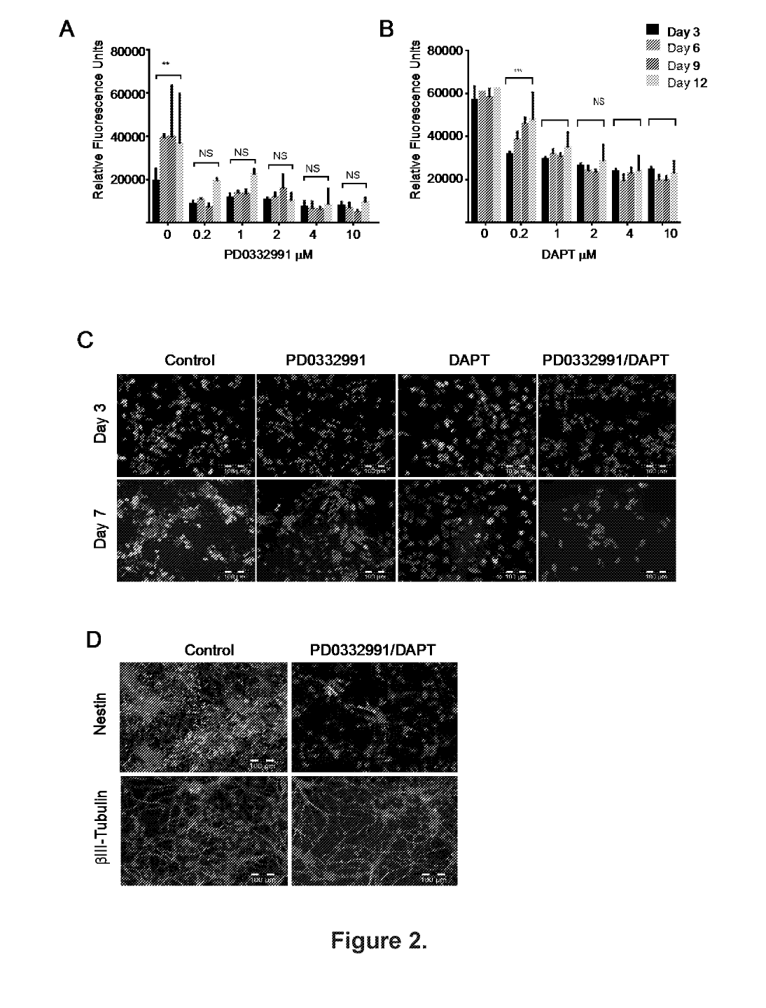

FIG. 2. PD0332991 and DAPT inhibit neural progenitor proliferation during differentiation.
A & B. Bar graphs showing relative fluorescence values (proportional to cell number), determined using the Cyquant cell proliferation assay. Cells were plated with increasing concentrations of PD0332991 (A) or DAPT (B) and assayed after 3, 6, 9 and 12 days of culture. Asterisks indicate significant increase in cell number over time ** p<0.05, ** p<0.01. NS indicates no significant increase in assay fluorescence resulting from growth inhibition by drug treatments.
C. Ki67 immunoreactivity (green) and cell nuclear staining with DAPI (blue) in neural progenitor cultures at 3 days (top panels) and 7 days (bottom panels) after plating in either control medium, or that supplemented with 2 μM PD0332991 or 10 μM DAPT, or both. Scale bars represent 100 micrometres, as indicated.
D. Nestin (top panels) and β-III-Tubulin (bottom panels) immunoreactivity of iPS-derived neural progenitors differentiated for 7 days in either control medium or that supplemented with 2 μM PD0332991 and 10 μM DAPT. Scale bars represent 100 micrometres, as indicated.

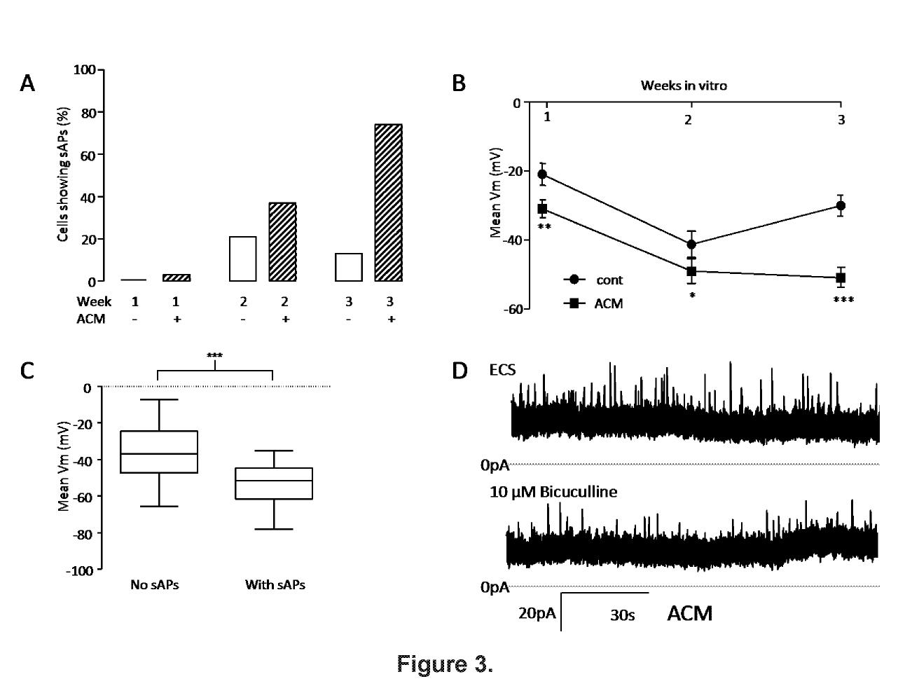

FIG. 3. Astrocyte conditioned medium enhances synaptogenesis, as measured by spontaneous electrical activity.
A. A bar graph showing the proportion of cells (%) at each week which fired (spontaneous action potentials (sAPs) during current-clamp (I=0 pA) at the native membrane potential (Vₘ). Chi² tests were performed at each week to compare control (ACM−) and ACM-treated (ACM+) iPS-derived neurons. ***P<0.0001, ^{ns}not significant; n=147.
B. Comparison of mean resting membrane potentials (Vₘ) of neurons differentiated in control medium (cont, circles) or ACM (squares) for up to 3 weeks. T-tests were performed at each week to compare control medium- and ACM-treated cells. *P<0.05, **P<0.01, ***P<0.001, ^{ns}not significant; n=147.
C. Box and whisker plots comparing all cells which either did, or did not, demonstrate spontaneous activity plotted against Vₘ. Horizontal bar is the mean, box shows the 95% confidence and whiskers show upper and lower quartiles. T-test was performed to compare the two means. ***P<0.001; n=298.
D. Example current recording during voltage-clamp (Vₕ=−20 mV) showing tetrodotoxin-insensitive spontaneous miniature inwards currents in cells differentiated for 3 weeks in ACM in absence (upper trace) and presence (lower trace) of GABA_{A} receptor blockade with 10 μM bicuculline.

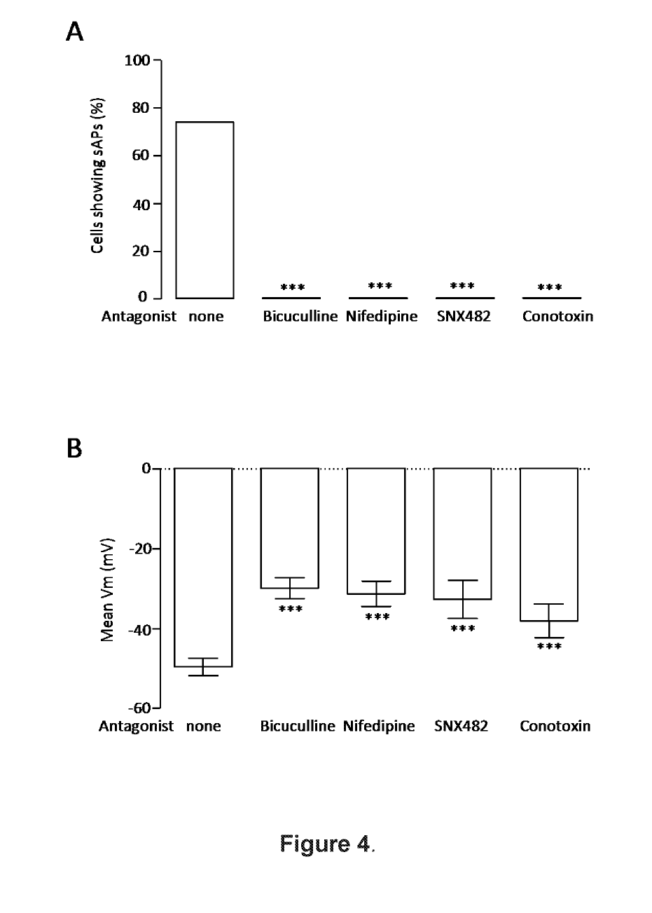

FIG. 4. Blockade of Ca²⁺ or GABA_{A} signaling occludes the synaptogenic effect of ACM. Bar graphs comparing the proportion of cells firing sAPs (A) and mean Vₘ (B) following 3 weeks of differentiation in ACM alone, or ACM with 10 μM bicuculline (a GABA_{A} antagonist), with 2 μM nifedipine (an L-type Ca²⁺ channel antagonist), with 100 nM conotoxin (a N-type Ca²⁺ channel toxin) or with 100 nM SNX482 (an R-type Ca²⁺ channel toxin). Chi² tests were performed comparing the ACM with each toxin against ACM alone. ^{±}P<0.05, ***P<0.001, ^{ns}not significant, n=57.

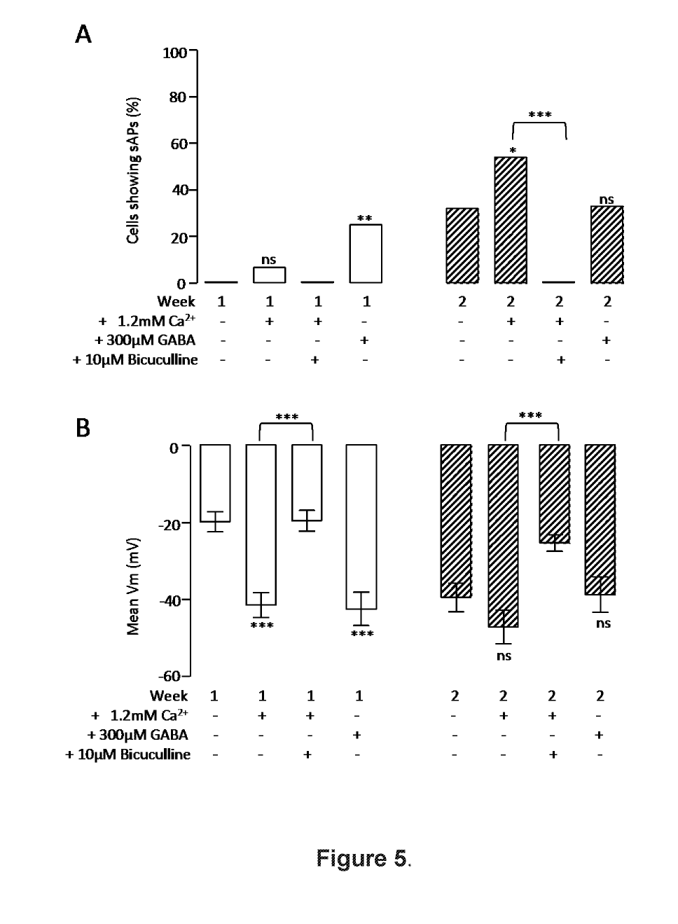

FIG. 5. High Ca²⁺ or GABA can partially mimic the effects of ACM, to promote hyperpolarization and spontaneous activity. Bar graphs comparing the proportion of cells firing sAPs (C) and mean Vₘ (D) following 1 and 2 weeks of differentiation in control medium (0.6 mM Ca²⁺), high Ca²⁺ medium (1.8 mM Ca²⁺), control medium with 300 μM GABA and, high Ca²⁺ medium with 10 μM bicuculline). T-tests were performed comparing the GABA and high Ca²⁺ media with control medium and the bicuculline medium with high Ca²⁺ medium for each week. ^{±}P<0.05, ***P<0.0001, ^{ns}not significant, n=133.

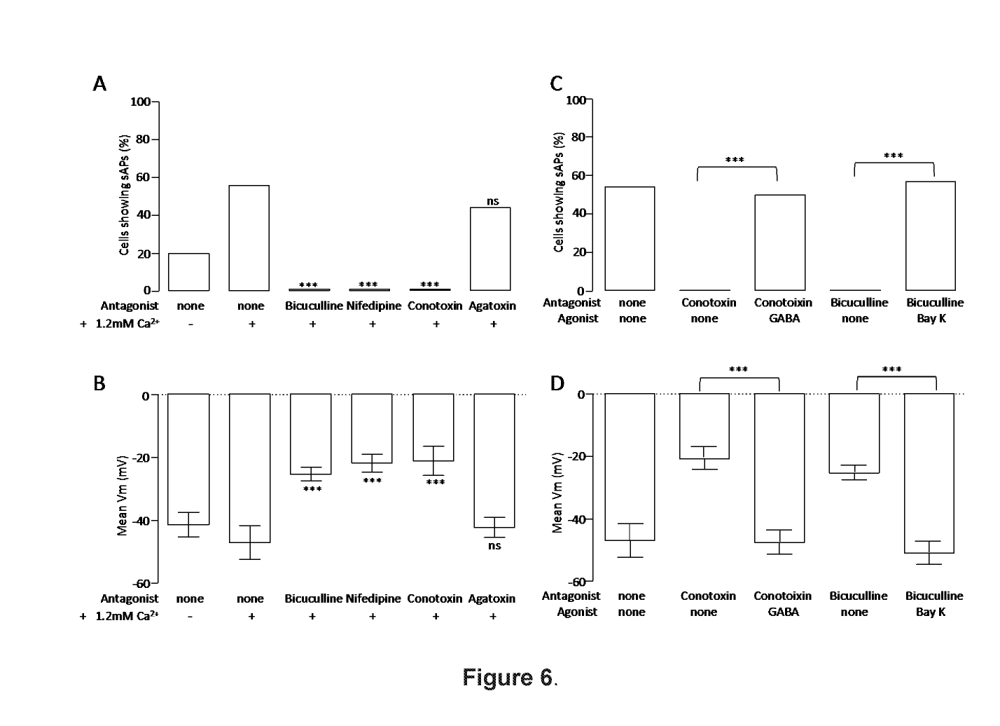

FIG. 6. Manipulation of GABA_{A} and Ca²⁺ currents in high Ca²⁺ medium: effects and functional rescue. Bar graph comparing the proportion of cells firing sAPs (A) and mean Vₘ (B) at week 2 when treated with control medium (0.6 mM Ca²⁺), high Ca²⁺ medium (1.8 mM Ca²⁺) and high Ca²⁺ medium with 10 μM bicuculline, with 2 μM nifedipine and with 100 nM conotoxin and, with 100 nM agatoxin (a potent P/Q-type Ca²⁺ channel toxin). Chi² tests were performed to compare the high Ca²⁺ medium with each toxin against high Ca²⁺ medium alone. ^{±}P<0.05, ***P<0.0001, ^{ns}not significant, n=90.

Bar graph comparing the proportion of cells firing sAPs (C) and mean Vₘ (D) at week 2 treated with high Ca²⁺ medium (1.8 mM Ca²⁺) and high Ca²⁺ medium with 100 nM conotoxin, conotoxin with 300 μM GABA, 10 μM bicuculline and, bicuculline with 1 μM BayK 8644 (an agonist of L-type Ca²⁺ channels). Chi² tests were performed to compare the proportion of cells with treated with conotoxin vs. conotoxin with GABA and biciculline vs. bicuculline with BayK 8644. ***P<0.0001, n=65.

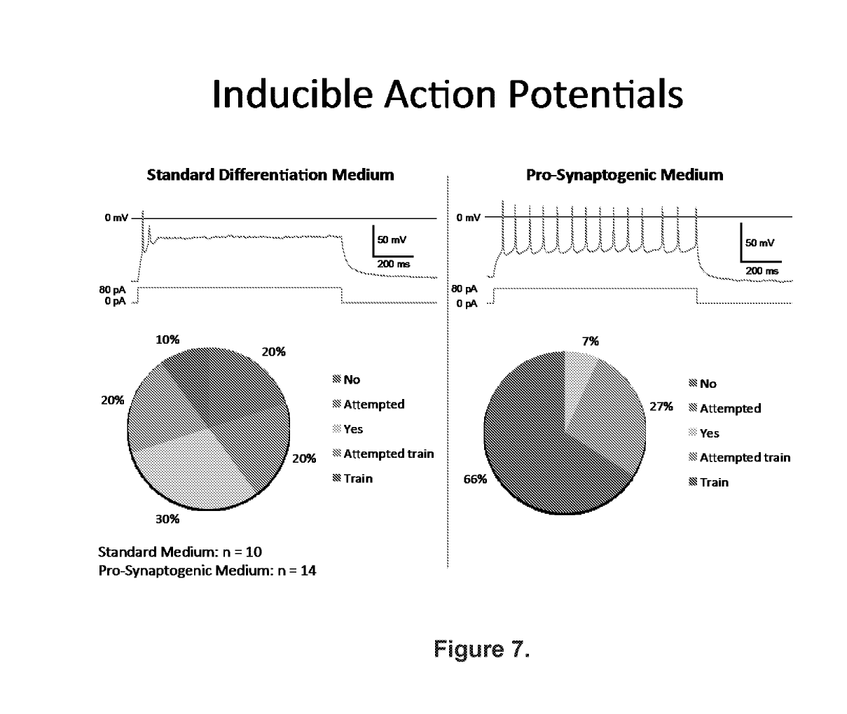

FIG. 7. The protocol dramatically enhances the ability of PSC-derived neurons to fire induced action potential trains. Inducible action potential generation in standard and pro-synaptogenic media: The percentage of cells displaying iAPs in the standard (SFDM4) and pro-synaptogenic (SCM1/2) medium (Cells plated from day 16 rosettes. SCM1/2, n=14, patched 21-23 days post rosette (DPR). SFDM4, n=10, patched 24 DPR) is shown, pie chart, bottom. Example traces shown above.

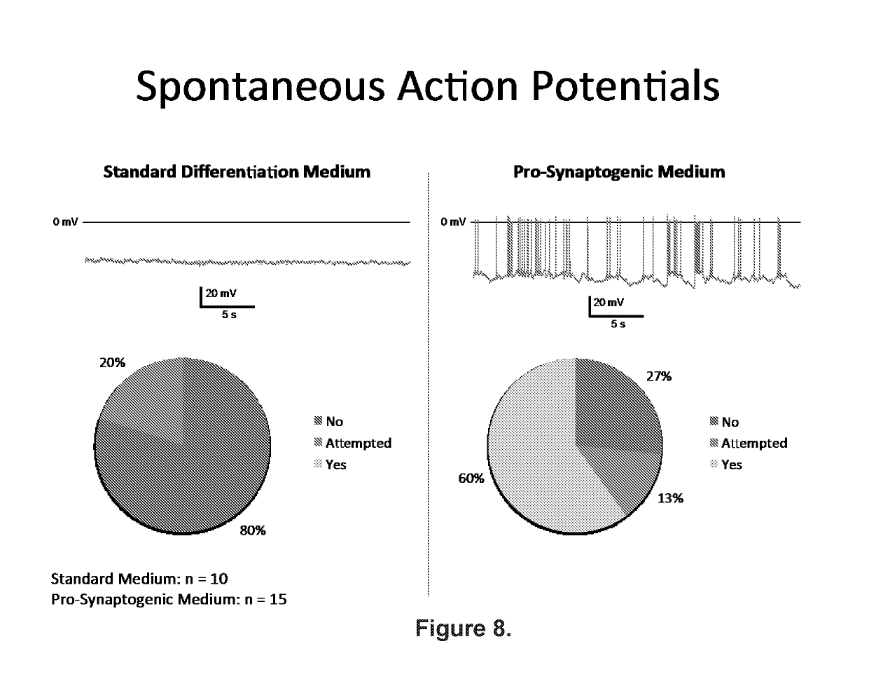

FIG. 8. The protocol dramatically enhances the spontaneous action potential activity of PSC-derived neurons. Spontaneous action potential generation in standard and pro-synaptogenic media: The percentage of cells displaying sAPs in the standard (SFDM4) and pro-synaptogenic (SCM1/2) medium (Cells plated from day 16 rosettes. SCM1/2, n=15, patched 21-23 DPR. SFDM4, n=10, patched 24 DPR) is shown, pie chart, bottom. Example traces shown in FIG. 8.

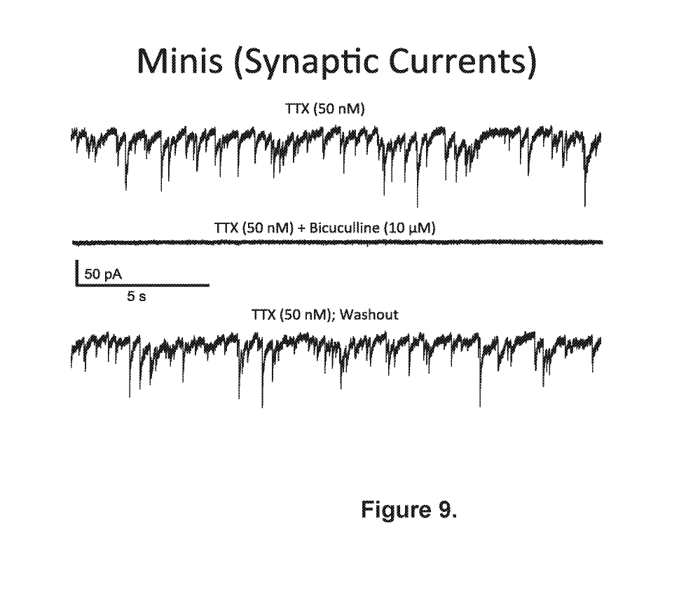

FIG. 9. The protocol promotes the development of high frequency of miniature synaptic currents. Example shows high frequency GABA_{A}R-dependent miniature synaptic currents in neurons differentiated by the protocol, as above. Miniature currents were measured in voltage-clamp (holding potential=−70 mV) in the presence of 50 nM TTX, and are indicative of spontaneous vesicular release of neurotransmitters at the synaptic cleft of networked neurons. These currents were entirely abolished by the addition of 10 μM bicuculline, a selective GABA_{A}R antagonist (n=5).

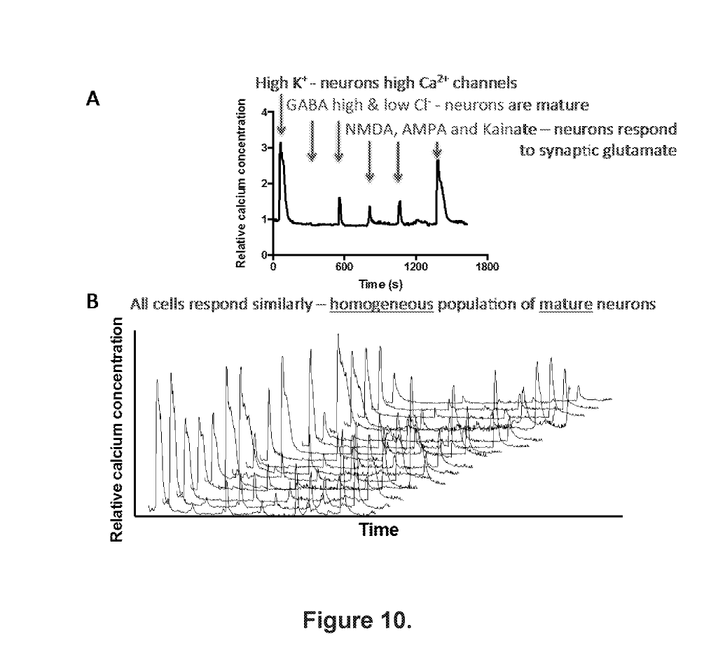

FIG. 10. The protocol generates functionally mature neurons with a high degree of homogeneity. Cells were plated from day 16 rosettes in SCM1/2, and were imaged at 21 days post rosette (DPR).

A. Exemplar time-course of Fura 2 fluorescence recording of intracellular Ca²⁺ concentration in a neuron derived from hPSCs differentiated using the protocol. At each arrow, the cell was exposed sequentially for 15 s to the following solutions: ECS with high potassium chloride concentration (50 mM, isoosmotically replacing NaCl) to evoke a cell depolarization; ECS containing 300 micoM GABA; ECS with low sodium chloride concentration (15 mM final, isoosmotically replaced by sodium isethionate) with 300 microM GABA, ECS containing 100 micromolar NMDA plus 10 micromolar glycine; ECS containing 100 micromolar AMPA, and: ECS containing 100 micromolar kainite.

B. Representation time-courses of Fura 2 fluorescence recording of intracellular Ca²⁺ concentration in all of the hPSC-derived neurons, differentiated using the protocol, within a field. The solution additions were exactly as described in panel A. Note that each recording has been moved to the right and upwards (pseudo 3-D view) in order to show clearly each trace.

Table 1. First neural differentiation medium components (SCM1) their function and alternative components thereof;

Table 2. Second neural differentiation medium components (SCM2) their function and alternative components thereof;

Table 3. Cell culture medium components and component concentrations;

Table 4. SCM1 Differentiation medium components and component concentrations;

Table 5. SCM2 Differentiation medium components and component concentrations.

### Methods

### Background to the Development of the Differentiation Protocol

The significance of high Ca²⁺ concentration and GABA addition were discovered whilst analysing the function of astrocyte conditioned medium (ACM) in promoting neuronal differentiation and the specific role of voltage-activated Ca²⁺ channels in said process. In these studies, neural progenitor cells were plated and differentiated as previously described in medium supplemented with B27 which had or had not been conditioned with mouse cortical astrocytes.

### Production of ACM

Confluent cultures at P1 and P2 were used to condition neural differentiation base medium (DMEM:F12 (1:3), 2% B27, 1% non-essential amino acids; all from Life Technologies, Paisley, Strathclyde, U.K.). ACM was harvested after 72 h, filtered sterilised, aliquoted and stored for later use at −80° C. Different batches of ACM were compared using a mouse CCL2 (chemokine C-C motif ligand 2) ELISA (Quantikine Mouse CCL2/JE/MCP-1 immunoassay. R & D Systems, Abingdon, Oxon., U.K.) and normalised to a constant final concentration of CCL2 of 1 mg·ml⁻¹. For differentiating neurons, ACM was mixed with differentiation base medium in a 1:1 ratio.

### The Differentation Protocol

The protocol is applied to the neuronal differentiation of neural progenitor cells. Neural progenitor cells may be obtained from a variety of sources, notably the neural induction of and differentiation of PSCs to Nestin/Pax6/Sox2-positive neural rosette stage progenitors. A cell suspension of neural progenitor cells conventionally derived from PSCs by various published protocols (freshly prepared or taken from frozen) are plated directly for neural differentiation. Cells may be plated onto glass coverslips, which may or may not be coated with one of any established coatings, including but not exclusively and in any combination, poly-D-lysine, matrigel, poly-L-lysine and laminin. Cells may also be plated onto tissue culture plastic.

For data presented in the attached figures, a single cell suspension (obtained by accutase treatment) of neural progenitors was plated at a density of ˜1×10⁸ cells/ml on poly-D-lysine/matrigel-coated coverslips within the wells of a 24-well tissue culture plate. Plated cells were allowed to adhere for 1 h at 37° C. and then cultured in 500 microlitres of SCM1 differentiation medium (neurogenic medium table 4) and the medium was exchanged by 80:20 replacement with fresh SCM1 differentiation medium 2 and 5 days after plating.

At 7 days post-plating, the medium was changed from SCM1 to SCM2 differentiation medium (synaptogenic medium table 5), and cell culture was continued in SCM2 differentiation medium, exchanging 80:20 of the SCM2 medium every 2-3 days, for up to 30 days. Those skilled in the art will appreciate one can culture differentiated cells beyond the day period if required.

### Cell Proliferation

Cell proliferation in cultures treated with cell cycle and Notch pathway inhibitors were measured using the Cyquant™ Direct Cell Proliferation kit (Invitrogen). Briefly, 1×10⁴ neural progenitors were plated per well in black, clear-bottomed 96 well plates (Nunclon) pre-treated with matrigel and cultured in ADF medium supplemented with B27 and small molecule inhibitors such as PD0332991 or DAPT (FIG. 2). Plates were processed according to the manufacturer's instructions and fluorescence Cyquant measurements were taken from triplicate cells on days 3, 6, 9 and 12 of culture using a FLUOstar Optima™ plate reader (BMG Labtech) using FITC (480 nm excitation/535 nm emission) filters in a bottom read fluorescence mode. Each measurement was calibrated by using blank suppression dye as a blank to remove background fluorescence.

### Immunocytochemistry

Cells grown on coverslips as described above were fixed with 4% paraformaldehyde at room temperature for 15 minutes. Cells were permeabilised (0.1% Triton X-100 in phosphate buffered saline) for 20 minutes at room temperature, then blocked in blocking solution (2% normal goat serum, 3% bovine serum albumin, 0.1% Triton X-100 in phosphate buffered saline for one hour. Cells were incubated with primary antibodies (rabbit anti-FOXG1, mouse anti-Sox2 (Abcam), mouse anti-ZOI (Abcam), mouse anti-Nestin (Abcam) anti-KI67 (Sigma) and anti-βIII-Tubulin (Sigma) diluted in the same buffer overnight at 4° C. Coverslips were then washed in 0.1% Triton X-100 in phosphate buffered saline 3 times for 5 minutes each and incubated with secondary antibodies (Goat anti-rabbit alexa 594 or Goat anti-mouse 488 secondary antibodies (1:400, Sigma), as appropriate diluted in blocking buffer. Coverslips were washed as above, mounted onto slides using Vectashield with DAPI and imaged using a Olympus BX61 fluorescence microscope.

### Physiological Characterisation of Neurons

### Whole-Cell Patch-Clamp

Extracellular Solution (ECS) contained in mM: 135 NaCl, 5 KCl, 5 HEPES, 10 Glucose, 1.2 MgCl₂, 1.25 CaCl₂, pH adjusted to 7.4 with NaOH. Intracellular Solution (ICS) contained (in mM): 117 KCl, 10 NaCl, 11 HEPES, 11 EGTA, 2 MgCl₂, 1 CaCl₂, 2 Na₂.ATP, pH adjusted to 7.2 with KOH. Ag/AgCl electrodes for patch-clamp were produced by leaving pure silver wire in bleach overnight. An Ag/AgCl pellet (Clark Electromedical Instruments) connected to the patch-clamp headstage was used as the bath (reference) electrode. Thin-walled, filamented, borosilicate glass tubes (World-Precision Instruments, UK) were pulled using a two-stage pipette puller (PP-830, Narishige, Japan) to give patch pipettes with a measured tip resistance of 3-6 MΩ when filled with the internal pipette solution. These pipettes were used to establish whole-cell configuration of the patch clamp technique.

Whole-cell patch-clamp was performed at room temperature. Plated down cells grown on glass coverslips were transferred into a perfusion chamber mounted on the stage of an inverted microscope equipped with phase-contrast optics mounted inside a Faraday cage on an anti-vibration air table (Wentworth Laboratories Ltd., Oxford, UK). Continuous perfusion of the cells with ECS was maintained by a gravity-fed system using a 50 mL disposable syringe as a reservoir. Continuous perfusion was maintained at a rate of approximately 2-4 ml per minute. A gravity-fed rapid solution changer (RSC, RSC-200, Bio-Logic, France) was used to apply various solutions directly to the cells when required allowing for solution changes as fast as 20 ms. Waste solution was removed via vacuum suction through a flame-pulled glass tube connected to a waste Buchner flask with Tygon tubing. Patch-clamp pipettes were partially filled with ICS and mounted on a CV 201AU headstage attached to an Axopatch 700A Amplifier (Axon Instruments; Forster City, Calif., USA) and DigiData 1320 A/D interface.

Current-clamp was used to apply current protocols, generated using Axon Laboratory's pClamp 9 software to the patched cell. These were recorded on-line for further analysis using the pClamp software. Offline data review and analysis was performed using Axon Laboratory's Clampfit 9, Microsoft Office Excel and Prism6. Spontaneous action potentials (sAPs): Cells were held in current clamp with zero current injection for recording of resting membrane potentials (Vm) and observation of any spontaneous action potentials. Recordings were made using the ‘Gap free’ protocol immediately after whole-cell access to reduce the effect of the pipette ICS dialysing with the actual cytosolic solution and thus influencing the resting membrane potential. Recordings were made for approx. 60 s for a mean calculation of Vm and to allow for spontaneous action potentials to be recorded.

Induced action potentials (iAPs): Variable current injection was used to hold the membrane potential close to −80 mV. A sequential injection of current for 1000 ms from 0 pA to 180 pA in 10 pA increments taking 19 sweeps was used in an attempt to elicit an action potential response upon the membrane potential reaching and passing the action potential threshold value.

Data from the current step protocols were analysed in Clampfit and exported to Excel for further processing. The average Vm during a 60 s ‘Gap Free’ recording in current clamp was taken as the resting membrane potential. In cells displaying spontaneous activity, an estimation of the baseline was made from the trace. For sAPs, resting membrane potential recordings in zero-current injection mode were categorised as follows: No—no apparent action potentials, Attempted—action potential-like events which failed to depolarise above 0 mV, and Yes—action potentials which depolarised above 0 mV. For iAPs, membrane voltage recordings during sequentially increasing current steps, from a holding current producing approx. −80 mV, were categorised as follows: No—no apparent action potentials, Attempted—action potential-like events which failed to depolarise above 0 mV, Yes—action potentials which depolarised above 0 mV, Attempted train—repeat action potential-like events following an initial action potential above 0 mV which fail to depolarise above 0 mV, and Train—repetitive action potentials following an initial action potential above 0 mV which depolarise above 0 mV.

### Ca²⁺ Imaging

The ratiometric Ca²⁺ sensitive dye Fura-2-AM (Molecular Probes, Eugene, Oreg., USA) was used for Ca²⁺ imaging experiments. This dye was purchased as a dry powder in 50 μg aliquots. Before use, these aliquots were dissolved in 50 μl DMSO (Sigma) by vortex. They were then incubated at 37° C. for 1 h to ensure the dye was fully dissolved. 1 μl of the 1 mg/ml Fura-2-AM stock was added to 250 μl of normal cell growth media and mixed. Cells on glass coverslips were then added to the well and incubated for 30 min at 37° C. and 5% CO₂ in a humidified tissue culture incubator. Both stocks and media containing the dye were stored out of direct light to prevent photo-bleaching of the dye. This incubation allowed for Fura-2-AM to be taken up by the cells, cleaved and turned into the active dye Fura-2. Fura-2-loaded cells were placed in a specialised perfusion chamber, designed to allow direct access of the microscope oil-immersion lens with the bottom of the coverslip, mounted upon an Olympus IX71 equipped with a Cairn monochromator-based fluorescence system (Cairn Instruments, Faversham, UK) and were continuously superfused with ECS. Imaging was performed at room temperature. Fura-2 was alternately excited using fluorescence light emitted from the monochromator at 340 and 380 nm. Images at 510 nm were acquired at 0.33 or 0.2 Hz by a slow-scan CCD camera (Kinetic Imaging Ltd, Nottingham, UK). Solutions, agonists and antagonists were locally applied to the cells using the gravity-driven rapid solution changer.

### Ca²⁺ Imaging Data Analysis

Raw data, in the form of emission intensities at 510 nm, during alternate excitation at 340 or 380 nm, were recorded and stored. Following background subtraction, emission ratios (340/380) were calculated off-line using Microsoft Office Excel (Microsoft, Redmond, Wash., US). Where indicated, data were normalised to the first 10 frames to allow further analysis and for comparisons.

### Results

Neural progenitors can be derived from pluripotent stem cells using a variety of protocols described in the literature, and these could be used for further neuronal differentiation using the protocols described herein. Neurogenesis and neuronal maturation is exemplified here using neural progenitors derived from hPSCs employing the protocol described in the legend of FIG. 1. Such neural progenitors are characterised as Foxg1, Sox2- and Nestin-positive cells, with a high incidence of neural rosette formation identified by foci of zona occludens-1 (ZO-1) immunoreactivity (FIG. 1) and a high mitotic index (see, for example, Ki67 immunoreactivity shown in FIG. 2 B, control).

A critical step in the transition from neural progenitor to neuron is the lengthening of the cell cycle leading to cell cycle exit. To develop culture medium to initiate neurogenesis we first aimed to synchronise progenitor cell cycle exit using two small molecules. First we used PD0332991, a small molecule inhibitor of the cyclin-dependent kinase 4/6 to block progression through the G1/S checkpoint and to hold neuroblasts in G1. FIG. 2A shows the concentration-dependent growth arrest in cultures treated with PD0332991. Forced cell cycle exit by treatment with PD0332991 is seen by the complete loss of Ki67 immunopositive cells after 3 days of treatment (FIG. 2C). In addition, we used gamma-secretase inhibitors (exemplified by DAPT) to block Notch pathway signalling. Notch pathway inhibition is known to activate proneural gene expression, and it also reduces cell proliferation (FIG. 2B). However DAPT treatment alone was insufficient to synchronise cell cycle exit and differentiation, as seen by the more progressive loss of Ki67 immunostaining compared to that seen in PD0332991 treated cultures (FIG. 2C). Thus, in combination, gamma secretase inhibition and cell cycle arrest resulted in synchronous and sustained cell cycle exit and enhancement of the proneurogenic “programme”. Neural differentiation in PD0332991/DAPT treated cultures after 7 days of treatment was seen by extensive expression of the neurofilament β-III-Tubulin, and a marked reduction in expression of the neural progenitor marker Nestin, compared to control cultures (FIG. 2D).

The use of astrocyte co-culture or astrocyte-conditioned medium (ACM) has been commonly employed to promote neuronal maturation in vitro. However, the mechanism of action of ACM are poorly defined, and critically, the constituents of ACM are completely undefined. As such, ACM cannot be used as a robust and reproducible reagent for stem cell research, where use of defined media are paramount. We observed that ACM promotes the functional maturation of PSC-derived neurons, as assessed by a more hyperpolarised resting membrane potential (FIG. 3A) and a large increase in the proportion of cells able to generate spontaneous action potentials (FIG. 3B); there was a clear correlation between resting membrane potential and demonstration of spontaneous activity (FIG. 3C). Furthermore, ACM promoted high levels of miniature synaptic potentials (FIG. 3D), indicative of the development of mature networks of synaptically connected neurons.

In order to understand how ACM might promote such functional maturation of PSP-derived neurons, differentiation in ACM was performed in the presence or absence of specific ion channel blockers. Thus, the GABAA receptor blocker, bicucculine, and the specific, voltage-activated Ca²⁺ channel antagonists, nifedipine, conotoxin and SNX-482 (which block L-, N- and R-type channels, respectively) all abolished the ACM-evoked induction of spontaneous activity (FIG. 4A) and hyperpolarization (FIG. 4B). These data suggest that ACM may be exerting its effect via regulation of GABA_{A} and Ca²⁺ signalling pathways.

To test the idea that Ca²⁺ influx was an important determinant of neuronal maturation, extracellular Ca²⁺ concentration was increased in the control medium from 0.6 mM to 1.8 mM and both resting Vₘ and spontaneous activity were again measured using whole cell patch-clamp. The increased Ca²⁺ concentration resulted in an enhancement in the proportion of cells generating spontaneous activity, which became significant at week 2 (FIG. 5A). Increased Ca²⁺ also evoked significant hyperpolarisation of mean resting Vₘ at week 1 (FIG. 5B). By week 2, the resting Vₘ of both groups had stabilized at this hyperpolarised level (FIG. 5B). These data suggested that increasing Ca²⁺ influx could partially mimic ACM. However, without a significant depolarizing stimulus, neither increased Ca²⁺ channel expression nor an increased driving force, alone or in combination, would necessarily result in increased intracellular Ca²⁺. Importantly, the ability of high extracellular Ca²⁺ to hyperpolarize the cell and augment spontaneous activity was completely ablated when GABA_{A} receptors were blocked by 10 μM bicuculline (FIG. 5A, C). Moreover, addition of 300 μM GABA was as effective as the increased extracellular Ca²⁺ at week 1 in augmenting spontaneous activity and hyperpolarising the Vₘ (FIG. 5A, C). These data show that GABA-dependent depolarisation or high extracellular Ca²⁺ are both capable of mimicking the ACM-evoked maturation of differentiating neurons.

Pertinent channel blockers were employed to test whether high Ca²⁺ might augment the maturation of neurons via GABA_{A}-dependent Ca²⁺ influx through specific Ca²⁺ channels. In this separate set of experiments, 2 weeks of differentiation in 1.8 mM Ca²⁺ promoted an increase in spontaneous activity and hyperpolarized the Vₘ (FIG. 6A, B). Importantly, in the presence of 1.8 mM Ca², bicuculline, nifedipine or conotoxin completely blocked spontaneous activity and depolarised the Vₘ (FIG. 6A, B). In contrast, agatoxin (a P/Q channel blocker) was completely without affect (FIG. 7A, B), suggesting that the effect of high Ca²⁺ was dependent upon L- and N-type Ca²⁺ channels and GABA signalling.

Since either GABA_{A} receptor or N-type Ca²⁺ channel blockade diminished the positive effects of high Ca²⁺ on functional maturation, attempts were made to bypass the effects of each by activating alternative Ca²⁺ influx mechanisms. Thus, conotoxin co-treatment with high Ca²⁺ completely blocked the ability of high Ca²⁺ to augment spontaneous activity (FIG. 6C) and depolarised the cells (FIG. 6D). Remarkably, the effect of conotoxin was reversed by GABA (FIG. 6C, D), which re-established the ability of cells to fire action potentials and evoked a hyperpolarisation. Equally striking was the ability of the L-type Ca²⁺ channel opener, Bay K8644, to rescue the bicuculline-evoked diminution of excitability (FIG. 6C, D).

These data suggest the rate and extent of functional neuronal maturation of PSC-derived neurons can be greatly augmented by stimulating GABA_{A} receptor-dependent Ca²⁺ influx through voltage-gated Ca²⁺ channels and provided the basis for our development of defined media for the maturation of hPSC-derived neurons.

Utilizing our novel background information (from FIG. 1-6), a new prosynaptic, in vitro PSC differentiation protocol was developed. Thus, at the neural rosette stage, neurogenesis is synchronised by forcing cell cycle exit, coupled to proneural gene activation, and in the presence of high calcium, pathways involving GABA_{A} receptor are activated. One consequence of GABA_{A} receptor activation is CREB phosphorylation which in turn plays a major role in regulating neurogenic gene expression. Importantly, sustained phospho-CREB activity is required during the presynaptic phase of neuronal differentiation. To maintain stable levels of CREB pathway activation during the initial neurogenic phase of the differentiation protocol medium was also supplemented with small molecule agonists of the pathway such as Forskolin (Table 2). In vivo, the early neurogenic phase of differentiation (characterised by tonic GABA stimulation and stable pCREB activity) is followed by a phase of activity-dependent maturation, driven by synaptogenesis. Since WNT signalling provides one of the major synaptogenic pathways differentiation media were also supplemented with the WNT pathway agonist CHIR99021.

The result of this protocol is to enhance greatly the rate and extent of maturation of PSC-derived neurons, as evidenced by its ability to promote the ability to fire repetitive trains of induced action potentials (FIG. 7), generate high levels of spontaneous activity (FIG. 8) and miniature synaptic potentials (FIG. 9) within 3 weeks, results which are unobtainable by standard neuronal differentiation protocols (FIGS. 7 and 8). Furthermore, this protocol also results in the generation of a homogenous population of mature neurons. Thus, in the Ca²⁺ imaging experiments shown in FIG. 10, all cells responded in an essentially similar manner. Specifically, the high potassium exposure demonstrated that the neurons contained voltage-gated Ca²⁺ channels. Although all neurons responded to GABA in high chloride, few cells responded to GABA in low chloride, indicating that even though all neurons expressed the machinery to depolarize in response to GABA (a marker of immature neurons), very few did so. In other words, the majority of neurons demonstrated a mature, post-natal phenotype. In addition, all cells responded similarly to NDMA, AMPA and kainite showing that they expressed all three excitatory, iontropic glutamate receptors.

### SUMMARY

Taken together, the protocol separates the two key phases of neural differentiation; the neurogenic phase and the synaptogenic phase, each characterised by different signalling requirements. These requirements have been met by formulation of two defined media that when used sequentially on plated PSC-derived neural progenitor cells lead to rapid neuronal differentiation and functional maturation.

TABLE 1

| Component | Rationale | Potential alternative reagents |
|---|---|---|
| Advanced DMEM:F12 | Standard DMEM:F12 medium | Similar chemically defined base |
| | with N2-like supplement | media available |
| NeuroBrew 21 | A recent modification of B27 | B27 supplements (Invitrogen) + |
| | supplement | other brands e.g. GS21 |
| | (Chen et al. (2008)Journal of | (Amsbio) |
| | Neuroscience Methods 171, 239- | |
| | 247) | |
| PD0332991 | CDK4/6 inhibitor | Small molecule inhibitors of cell |
| | Small molecule inhibitor of cell | cycle progression that hold cells |
| | proliferation targeting the G1/S | in G1 of the cell cycle |
| | checkpoint and forcing cell cycle | |
| | exit | |
| BDNF | Recombinant protein | BDNF mimetics. |
| | neurotrophin TRKB agonist. | Although BDNF is sufficient |
| | Neurotrophic support. | GDNF, NGF, NT3 and NT4 may |
| | | also provide neurotrophic support |
| LM22A4 | Small molecule TRKB agonist | BDNF mimetics. Alternative small |
| | | molecule TRKB agonists e.g. 7,8- |
| | | Dihydroxyflavone |
| CHIR 99021 | GSK3β inhibition. Canonical WNT | Several alternative GSK3β |
| | signalling pathway activation is | inhibitors available (e.g. Bio, 3F8, |
| | pro-synaptogenic | A1070722). |
| CaCl₂ | Sufficiently high extracellular Ca²⁺ | |
| | is required to drive GABA induced | |
| | Ca²⁺ entry | |
| Ascorbic acid | Vitamin C | |
| GABA | Endogenous GABAA agonist | Alternative GABA analogues |
| | | available. |
| | | Ca²⁺ entry can be achieved by |
| | | direct voltage gated calcium |
| | | channel opening (e.g. by BayK |
| | | 8644) |
| DAPT | γ-secretase inhibitor. | Small molecules that inhibit |
| | Notch pathway inhibition | Notch. Multiple other γ-secretase |
| | promotes cell cycle exit and | inhibitors are available some of |
| | regulates proneurogenic gene | which we have also tested (e.g. |
| | expression | Semagacestat (LY450139), BMS- |
| | γ-secretase inhibition is limited to | 299897, Avagacestat (BMS- |
| | first neurogenic phase of | 708163)). |
| | differentiation, being removed | |
| | from SCM2 to avoid adverse | |
| | effects in mature neurons | |
| Forskolin | Adenylyl cyclase activator | CREB phosphorylation and |
| | induces CREB phosphorylation | pathway activation can be |
| | and pathway activation | achieved by several alternative |
| | | means. cAMP analogues can be |
| | | used (e.g Dibutyryl-cAMP, 8- |
| | | Bromo-cAMP) or several |
| | | inhibitors for phophodiesterase |
| | | PDE4 are available (e.g. |
| | | Rolipram, _Ro 20-1724, ICI 63197, |
| | | CP 80633) that elevate cAMP. |

TABLE 2

| Component | Rationale | Potential alternative reagents |
|---|---|---|
| Advanced DMEM:F12 | Chemically defined base medium | Multiple sources of DMEM:F12 |
| | comprising standard DMEM:F12 | media and N2 supplements |
| | medium with N2 supplement | available |
| Neurobasal A | Chemically defined base medium | Similar chemically defined base |
| | for neuronal cell culture | media for neuronal culture |
| NeuroBrew 21 | A recent modification of B27 | B27 supplements (Invitrogen) + |
| | supplement | other brands e.g, GS21 |
| | (Chen et al. (2008)Journal of | (Amsbio) |
| | Neuroscience Methods 171, 239- | |
| | 247) | |
| PD0332991 | CDK4/6 inhibitor | Small molecule inhibitors of cell |
| | Small molecule inhibitor of cell | cycle progression that hold cells |
| | proliferation targeting the G1/S | in G1 of the cell cycle |
| | checkpoint and forcing cell cycle | |
| | exit | |
| BDNF | Recombinant protein | BDNF mimetics. |
| | neurotrophin TRKB agonist. | Although BDNF is sufficient |
| | Neurotrophic support. | GDNF, NGF, NT3 and NT4 may |
| | | also provide neurotrophic support |
| LM22A4 | Small molecule TRKB agonist | BDNF mimetics. Alternative small |
| | | molecule TRKB agonists e.g. 7,8- |
| | | Dihydroxyflavone |
| CHIR 99021 | GSK3β inhibition. Canonical WNT | Several alternative GSK3β |
| | signalling pathway activation is | inhibitors available (e.g. Bio, 3F8, |
| | pro-synaptogenic | A1070722). |
| CaCl₂ | Sufficiently high extracellular Ca²⁺ | |
| | is required to drive GABA induced | |
| | Ca²⁺ entry | |
| Ascorbic acid | Vitamin C | |
| Phorbol 12-myristate | Non-canonical WNT pathway | Alternative PKC activators (e.g |
| 13-acetate (PMA) | activation is pro-synaptogenic. | Phorbol 12,13-dibutyrate, |
| | Intracellular signalling pathways | bryostatin, cholesteryl sulphate) |
| | less well defined but include | |
| | signalling through PKC | |

TABLE 3
Advanced DMEM-F12 (ADF)

| Components | Amount | Company (Cat #) |
|---|---|---|
| Advanced DMEM/F12 (1:1) | 500 ml | Invitrogen (12634-010) |
| L-Glutamine (200 mM) | 2 mM (5 ml) | Invitrogen (25030-024) |
| Penicillin/Streptomycin | (5 ml) | Invitrogen (15070-063) |
| (5000 U/5000 μg) | | |

TABLE 4
SCM1

| Components | Amount/50 ml | | | Company (Cat #) |
|---|---|---|---|---|
| ADF | 49 | ml | | (prepared as |
| | | | | above) |
| NeuroBrew | 1 | ml | 2% (Similar to | Miltenyi |
| 21 | | | B27 + RA) | (130093566) |
| PD0332991 | 2 | μM | 10 μl (10 mM stock) | Tocris (4786) |
| DAPT | 10 | μM | 50 μl (10 mM stock) | Tocris (2634) |
| BDNF | 10 | ng/ml | 5 μl(100 ug/ml stock) | Miltenyi |
| | | | | (130096286) |
| Forskolin | 10 | μM | 50 μl (10 mM stock) | Tocris (1099) |
| CHIR 99021 | 3 | μM | 25 μl (6 mM stock) | Tocris (4423) |
| GABA | 300 | μM | 50 μl (300 mM stock) | Tocris (0344) |

| | | | |
|---|---|---|---|
| CaCl₂ | Final | 37 μl (1M stock) | Sigma |
| | 1.8 mM | | (499609-1G) |
| Ascorbic | 200□M | 10 μl (1M stock) | Sigma (A4544) |
| acid | | | |

TABLE 5
SCM2

| Components | Amount/50 ml | | | Company (Cat #) |
|---|---|---|---|---|
| ADF | 24.5 | ml | | (prepared as |
| | | | | above) |
| Neurobasal | 24.5 | ml | | Invitrogen |
| A | | | | (101888), |
| | | | | prepared as |
| | | | | for ADF) |
| NeuroBrew | 1 | ml | 2% (Similar to | Miltenyi |
| 21 | | | B27 + RA) | (130093566) |
| PD0332991 | 2 | μM | 10 μl (10 mM stock) | Tocris (4786) |
| BDNF | 10 | ng/ml | 5 ul (100 ug/ml stock) | Miltenyi |
| | | | | (130096286) |
| CHIR 99021 | 3 | μM | 25 μl (6 mM stock) | Tocris (4423) |
| CaCl₂ | Final | | 18.5 μl (1M stock) | Sigma |
| | 1.8 mM | | | (499609-1G) |
| Ascorbic | 200□M | | 10 μl (1M stock) | Sigma (A4544) |
| acid | | | | |
| PMA | 10 | nM | 0.5 μl (1 mM stock) | Sigma (8139) |

1. A neural cell differentiation medium for the production of neural cells from at least one precursor cell capable of lineage specific differentiation, comprising:
i) at least one cell culture base medium;
ii) at least one serum-free supplement for neural cell culture;
iii) a cell-cycle inhibitor;
iv) at least one Tropomyosin receptor kinase B [TrkB] receptor agonist;
v) a canonical Wnt pathway agonist;
vi) ascorbic Acid or vitamin C or a salt or derivative thereof; and
vii) a solution of calcium ions.

2. The neural cell differentiation medium according to claim 1 further comprising at least any one or more of the following, including all combinations thereof:
viii) a notch signaling inhibitor;
ix) a CREB (cAMP response element-binding protein) activator;
x) a γ-aminobutyric acid (GABA) receptor agonist; and
xi) a non-canonical WNT pathway agonist.

3. The neural differentiation medium according to claim 1 wherein said cell culture base medium is selected from the group consisting of: DMEM and DMEM:F12.

4. The neural differentiation medium according to claim 1 wherein said serum free supplement is selected from the group consisting of: N2 supplement, B27 supplement, glutamine supplement, and NeuroBrew™.

5. The neural differentiation medium according to claim 1 wherein said cycle inhibitor is an inhibitor of the G1/S cell checkpoint.

6. The neural differentiation medium according to claim 5 wherein said cell cycle inhibitor is selected from the group consisting of: PD0332991, AZD5438, Baicalein, CI 898 trihydrochloride, Daidzein, WYE 687 dihydrochloride, YC 1, flavopiridol, indisulam, purvalanol, seliciclib, mimosine, and purvalanol.

7. The neural differentiation medium according to claim 1 wherein said Tropomyosin receptor kinase B [TrkB] receptor agonist is Brain Derived Neurotrophic Factor (BDNF), neurotrophin-4 (NT-4), Glial cell-derived neurotrophic factor (GDNF), neurotrophin-3 (NT-3), or an analogue thereof.

8. The neural differentiation medium according to claim 7 wherein said Tropomyosin receptor kinase B [TrkB] receptor agonist is selected from the group consisting of: 7,8-dihydroxyflavone, N,N′,N″Tris(2-hydroxyethyl)-1,3,5-ben-zenetricarboxamide (LM22A4), and 4′-dimethylamino-7,8-dihydroxyflavone.

9. The neural differentiation medium according to claim 1 wherein said canonical Wnt pathway agonist is a Glycogen synthase kinase 3 beta (GSK3β) inhibitor.

10. The neural differentiation medium according to claim 9 wherein said canonical Wnt pathway agonist is selected from the group consisting of: CHIR99021, (2′Z,3′E)-6-Bromoindirubin-3′-oxime (BIO), (2′Z,3′E)-6-Bromoindirubin-3′-acetoxime (BIO-acetoxime), 5-Ethyl-7,8-dimethoxy-1H-pyrrolo[3,-4-c]isoquinoline-1,3(2H)-dione (3F8), 1-(7-Methoxyquinolin-4-yl)-3-[6-(tr-ifluoromethyl)pyridin-2-yl]urea (A1070722), N-[(4-Methoxyphenyl)methyl]-N′-(5-ni-tro-2-thiazolyl)urea (AR-A 014418), 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrole-2,5-dione (SB415286), N-(3-Chloro-4-methylphenyl)-5-(4-ni-trophenyl)-1,3,4-oxadiazol-2-amine (TC-G24), 2-Methyl-5-[3-[4-(methylsulfinyl)ph-enyl]-5-benzofuranyl]-1,3,4-oxadiazole (TCS2002), and 3-[[6-(3-Aminophenyl)-7H-pyrrolo[2,-3-d]pyrimidin-4-yl]oxyphenol ditrifluoroacetate (TWS 119).

11. The neural differentiation medium according to claim 2 wherein said notch signaling inhibitor is a γ-secretase inhibitor.

12. The neural differentiation medium according to claim 11 wherein said notch signaling inhibitor is selected from the group consisting of: N-[N-(3,5-Difluorophenacetyl)-L-alanyl]-S-phenylglycine t-butyl ester (DAPT), Semagacestat, BMS-299897, BMS-708163, dibenzazepine, MK-0752, RO4929097, LY900009, LY3039478, LY411575, MRK-003, MRK-006, and BMS-906024.

13. The neural differentiation medium according to claim 2 wherein said CREB activator is selected from the group consisting of: an adenylyl cyclase activator, cAMP or an analogue thereof, and a phosphodiesterase inhibitor.

14. The neural differentiation media according to claim 13 wherein said adenylyl cyclase activator is selected from the group consisting of: forskolin, N,N-Dimethyl-(3R,4aR,5S,6aS,10S,10a-R,10bS)-5-(acetyloxy)-3-ethenyldodecahydro-10,10b-dihydroxy-3,4a,7,7,10a-pentamethyl-1-oxo-1H-naphth-o[2,1-b]pyran-6-yl ester β-alanine hydrochloride, Pituitary Adenylate Cyclase-Activating Polypeptide 1-27, 1,9-Dideoxyforskolin, 5-[[2-(6-Amino-9H-purin-9-yl)ethyl]amino]-1-pentanol, and Colforsin dapropate hydrochloride.

15. The neural differentiation medium according to claim 13 wherein said cAMP analogue is selected from the group consisting of: Dibutyryl-cAMP, 8-(4-Chlorophenylthio)-2′-O-methylad-enosine-3′,5′-cyclic monophosphate acetoxymethyl ester, 8-Bromo-cAMP, and (S)-Adenosine, cyclic 3′,5′-(hydrogenphosphorothioate) triethylammonium.

16. The neural differentiation medium according to claim 13 wherein said phosphodiesterase inhibitor is selected from the group consisting of: CGH 2466 dihydrochloride, Cilostamide, Cilostazol, 5-[3-[(1S,2S,4R)-Bicyclo[2.2.1]hept-2-yloxy]-4-methoxyphenyl]tetrahydro-2(1H)-pyrimid-inone, Dipyridamole, EHNA hydrochloride, Enoximone, Etazolate hydrochloride, Gisadenafil besylate, MDL 12330A hydrochloride, Mesopram, Rolipram, Pentoxifylline, Anagrelide hydrochloride, N,N,2-Trimethyl-5-nitro-benzenesulf-onamide, 4-[(2R)-2-[3-(Cyclopentyloxy)-4-met-hoxyphenyl]-2-phenylethyl]-pyridine hydrochloride, Vinpocetine, Zaprinast, and Zardaverine.

17. The neural differentiation medium according to claim 2 wherein said γ-aminobutyric acid (GABA) receptor agonist is selected from the group consisting of: recombinant γ-aminobutyric acid (GABA), Calcium acetylhomotaurinate, 1-(4-Aminobutanoyl)-4-[1,3-bis(dihy-droxyphosphoryloxy)propan-2-yloxy]-7-nitroindoline-, Isoguvacine hydrochloride, 3-(2,5-Difluorophenyl)-7-(1,1-dimet-hylethyl)-6-[(1-methyl-1H-1,2,4-triazol-5-yl)metho-xy]-1,2,4-triazolo[4,3-b]pyridazine, MK0343, MRK 016, Muscimol, trans-4-Aminocrotonic acid, 5-Nitro-α-oxo-N-(1R)-phenylethyl]-1H-indole-3-acetamide, 2′,4-Difluoro-5′-[8-fluoro-7-(1-hydro-xy-1-methylethyl)imidazo[1,2-a]-pyridin-3-yl]-[1,1-′-biphenyl]-2-carbonitrile, and (Z)-3-[(Aminoiminomethyl)thio]prop-2-enoic acid sulfate.

18. The neural differentiation medium according to claim 2 wherein said non-canonical Wnt pathway agonist is a Protein Kinase C (PKC) activator.

19. The neural differentiation medium according to claim 18 wherein said non-canonical Wnt pathway agonist is selected from the group consisting of: Phorbol 12-myristate 13-acetate (PMA), Phorbol 12,13-dibutyrate, cholesterol sulphate, Bryostatin 1, 2-[(2-Pentylcyclopropyl)methyl]cycl-opropaneoctanoic acid (FR 236924), (2S,5S)-1,2,4,5,6,8-Hexahydro-5-(hy-droxymethyl)-1-methyl-2-(1-methylethyl)-3H-pyrrolo-[4,3,2-gh]-1,4-benzodiazonin-3-one, Ingenol 3-angelate, and 5-Chloro-N-heptylnaphthalene-1-sulf-onamide, 5-Chloro-N-(6-phenylhexyl)-1-naphth-alenesulfonamide.

20. The neural differentiation medium according to claim 1 wherein any one or more of iii)-v) is a small molecule compound or an isolated or recombinant peptide, protein or antibody.

21. The neural differentiation medium according to claim 1 wherein said solution of chemical ions is calcium chloride.

22. An ex vivo culture medium comprising the neural differentiation medium according to claim 1.

23. An ex vivo culture medium comprising the neural differentiation medium according to claim 2.

24. A method for neural cell differentiation comprising: culturing in a culture medium at least one precursor cell capable of lineage specific differentiation wherein said culture medium comprises a neural cell differentiation media according to claim 1 and following culture in said medium said precursor cell develops into a neural cell.

25. The method according to claim 24 wherein said precursor cell is selected from the group consisting of: stem cells; pluripotent or multipotent progenitor cells; embryonic stem cells (ESC); induced pluripotent stem cells (iPSC); adult stem cells; somatic stem cells; cancer stem cells; and any cell capable of lineage specific differentiation into a neural cell.

26. The method according to claim 24 wherein said neural cell is selected from the group comprising: any cell type classified as part of the nervous system; neurones; astrocytes; and glial cells.

27. The method according to claim 24 wherein said method comprises:
culturing said cell(s) in a first neural cell differentiation medium according to claim 2 wherein said medium comprises or consist of i-x;
followed by culturing said cell(s) in a second neural cell differentiation medium according to claim 2 wherein said medium comprises or consist of i-vii and xi.

28. The method according to claim 27 wherein said method comprises culturing said cell(s) in said first neural cell differentiation medium for a number of days selected from the group consisting of the following days 1, 2, 3, 4, 5, 6, 7, 8, 9, 10, 11, 12, 13, 14, 15, 16, 17, 18, 19 and 20 days.

29. The method according to claim 27 wherein said method comprises culturing said cell(s) in said second neural cell differentiation medium for a number of days selected from the group consisting of the following days 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 days.

30.-31. (canceled)

32. A kit for the differentiation of at least one precursor cell capable of lineage specific differentiation into a neural cell comprising:
a. a neural cell differentiation medium comprising:
i. at least one cell culture base medium;
ii. at least one serum-free supplement for neural cell culture;
iii. a cell-cycle inhibitor;
iv. at least one Tropomyosin receptor kinase B [TrkB] receptor agonist;
v. a canonical Wnt pathway agonist;
vi. ascorbic acid or vitamin C or a salt or derivative thereof;
vii. a solution of calcium ions;
viii. a notch signaling inhibitor;
ix. a CREB (cAMP response element-binding protein) activator;
x. a γ-aminobutyric acid (GABA) receptor agonist;
b. a neural cell differentiation medium comprising:
i. at least one cell culture base medium;
ii. at least one serum-free supplement for neural cell culture;
iii. a cell-cycle inhibitor;
iv. at least one Tropomyosin receptor kinase B [TrkB] receptor agonist;
v. a canonical Wnt pathway agonist;
vi. ascorbic acid or vitamin C or a salt or derivative thereof;
vii. a solution of calcium ions; and
xi) a non-canonical WNT pathway agonist; and
optionally, reagents and instructions pertaining to the use of said neural differentiation mediums.

33. The neural differentiation medium according to claim 2 wherein any one or more of viii)-xi) is a small molecule compound or an isolated or recombinant peptide, protein or antibody.
